(19) United States
(12) Patent Application Publication
(10) Pub. No.: US 2005/0004579 A1
(43) Pub. Date: Jan. 6, 2005

(54) Computer-assisted manipulation of catheters and guide wires

(76) Inventors: M. Bret Schneider, Menlo Park, CA (US); David J. Mishelevich, Playa del Rey, CA (US)

Correspondence Address: RITTER, LANG & KAPLAN, 12930 SARATOGA AE. SUITE D1, SARATOGA, CA, 95070, US

(21) Appl. No.: 10/608,862

(22) Filed: Jun. 27, 2003

(51) Int. Cl. A61F 11/00

(52) U.S. Cl. 606/108

(57) ABSTRACT

Computer-assisted methods and systems for manipulating elongate members during medical procedures are provided. Electromechanical devices are computer driven to manipulate the elongate members, which a user can direct through computer interfaces like graphical user interfaces and pointing devices.

### BACKGROUND OF THE INVENTION

The present invention relates to computer-assisted manipulation of elongate (flexible or rigid) members while performing medical procedures or surgery. More specifically, the invention relates to computer-assisted manipulation of catheters and guide wires during medical procedures.

There are numerous kinds of minimally surgical procedures that require the manipulation of elongated elongate members through areas of a patient's body. A common example is angioplasty that attempts to increase blood through a blocked artery. The elongated elongate members can include guide catheters, interventional catheters (e.g., angioplasty or stent), guide wires, and the like. Typically, such procedures are performed by physicians who are in the fields of cardiology, radiology, and neurosurgery.

FIG. 1 shows an illustration of a common medical procedure such as angioplasty. An incision is made to reach a large artery, such as incision 1 in the femoral artery in a patient's leg. Other arteries such as in the arm can also be utilized. Elongate members 3 are inserted in the artery and manually directed up to a targeted lesion 5 for the desired procedure.

For example, elongate members 3 can include a guide catheter, a guide wire, an angioplasty balloon catheter, an angioplasty stent catheter, and an atherectomy catheter. The angioplasty balloon catheter, an angioplasty stent catheter, and an atherectomy catheter are examples of what will be called “interventional catheters.” In a typical angioplasty procedure, the guide wire is inserted in the guide catheter which is then manually steered through the arteries to the coronary os 7. The guide wire is then advanced to targeted lesion 5.

The angioplasty catheter is then inserted in the guide catheter but around the guide wire. In this manner, the angioplasty catheter follows the path of the guide catheter and guide wire to targeted lesion 5. Once at the lesion, a balloon on the angioplasty catheter may be inflated in order to dilate the lumen and thereby increase blood flow.

Other interventional catheters besides the angioplasty catheter can also be used. For example, a stent catheter can be utilized to insert a stent at targeted lesion 5 in order to increase blood flow. Physicians often utilize a combination of interventional catheters in order to achieve the desired results.

Although these medical procedures have met with great success, the current techniques for performing these procedures have numerous disadvantages. A major drawback to the use of elongate members in the minimally invasive surgical environment from the perspective of the physician is significant exposure to radiation due to the fluoroscopic cameras used to visualize the path and tip of guide wires, guide catheters, balloon catheters, biopsy needles, and other minimally invasive instruments. Lead-lined garments are typically worn by physicians, nurses and technicians during such procedures to reduce radiation exposure. However, the use of such garments itself presents an occupational hazard due to the wear and tear inflicted on the user's body, secondary to the weight and stiffness, and reduced ventilation that characterize such wearable radiation shields. Additionally, the arms and head of the user are not typically covered by such garments, making radiation shield at best incomplete.

Another disadvantage is that the elongate members are quite long and difficult to manually manipulate. For example, the length of the typical elongate members can be as follows:
Guide catheter—100 cm
Balloon catheter—130 cm
Stent catheter—135 cm
Guide wire—300 cm
   It can often require the cooperation of two or more individuals to manually manipulate these long elongate members in order to perform a procedure.

Additionally, it can be very difficult to manually maneuver the elongate members to their desired destination, manipulating a precise portion of the device to an exactly targeted spot within the body. This difficulty can be compounded by the complication that when a physician retracts one of the elongate members, such as to use a different elongate member, the other elongate members may be accidentally drawn from their desired location. This can require the elongate members to once again be directed to their desired location.

It would be desirable to have innovative techniques by which persons performing a minimally invasive procedure under fluoroscopic visualization could move and steer elongate members to their intended location without suffering radiation exposure in the process. It would also be beneficial to provide a computer-assisted system that allows a physician to direct, manipulate and retract the elongate members in a more efficient manner.

### SUMMARY OF THE INVENTION

The present invention provides techniques for the computer-assisted, remote control manipulation of elongate members such as catheters and guide wires in medical procedures. For example, a computer system can utilize electromechanical devices in order to manipulate the elongate members. A user can select how she desires to manipulate the elongate members through a graphical user interface. Additionally, the user can direct the manipulation of the elongate members through a pointing device such as a joystick.

Advantages of the invention can include the ability to manipulate minimally invasive surgical tools while under fluoroscopic visualization by remote control, virtually eliminating radiation exposure and permitting, if desired, the operating physician and the patient being separated by great geographical distances. Other advantages of the invention can include that fewer individuals may be required to manipulate the elongate members, the elongate members may be manipulated in a more efficient manner (e.g., to the desired destination) and elongate members can be exchanged without affecting the position of the other elongate members.

Other functions related to a procedure may also be remotely controlled by the hardware and software interfaces. Such functions include the motorized advancement or retraction of a syringe plunger, so as to inject contrast media, flush the catheter with saline or heparin, or to draw a sample of blood for a blood gas analysis. Additionally, external equipment, such as intravascular arterial measurement devices may be triggered via specific buttons or other controls on the software or hardware interfaces of the invention. Some specific embodiments of the invention are described below.

In one embodiment, the invention provides a method of manipulating an elongate member during a medical procedure. Input is received from a user to manipulate the elongate member. Signals are sent to advance the elongate member if the input directs advancement of the elongate member. Signals are sent to retract the elongate member if the input directs retraction of the elongate member. And, signals are sent to rotate the elongate member if the input directs rotation of the elongate member.

In another embodiment, the invention provides an apparatus for manipulating elongate members during medical procedures. A base is coupled to an elongate member, the base being rotatable along an axis parallel to the elongate member. A first motor is coupled to the base that advances or retracts the elongate member along the axis. And, a second motor is coupled to the base that rotates the base, whereby the elongate member is rotated around the axis.

In another embodiment, the invention provides a method of manipulating an elongate member during a medical procedure. Two elongate members are retracted where a first elongate member is within the lumen of a second elongate member. The first elongate member is advanced relative to the second elongate member. In some embodiments, the first elongate member is advanced to substantially counter retraction caused by the retraction.

In another embodiment, the invention provides an apparatus for manipulating elongate members during medical procedures. A drum is coupled to two elongate members where a first elongate member is within the lumen of a second elongate member, the drum being rotatable along an axis perpendicular to the two elongate members and comprising a clip to retain the second elongate member such that when the drum rotates, the second elongate member is retracted along a first direction. A wheel is coupled to the drum such that the rotation of the drum also rotates the wheel and the first elongate member is retracted along the first direction, wherein the wheel rotates to advance the first elongate member along a second direction opposite the first direction.

Because the invention allows minimally invasive procedures done under fluoroscopic visualization to be done by remote control, the physician may stand or sit at a distance from radiation-emitting fluoroscopic cameras. For example, the physician operator can work within a compact area placed behind a freestanding transparent radiation shield, such as those standard in angioplasty catheterization laboratories. Behind the screen, but in front of where the operator sits or stands, can be a compact control panel having a computer, joystick and fluoroscopic display. The operator may step out from the protective screen while fluoroscopic cameras are off to perform manual adjustments to the equipment setup. For example, the operator, without need for fluoroscopic, radiation-producing visualization, may manually change catheters or wires mounted on the system. Likewise, clips may be manually fastened or unfastened, and catheters and wires may be manually shifted from one channel to another on the wheels of the module base. Alternatively, other personnel, such as nurses or technicians, may be called to do such manual tasks.

Other features and advantages of the invention will become readily apparent upon review of the following description in association with the accompanying drawings.

### BRIEF DESCRIPTION OF THE DRAWINGS

FIG. 1 shows an illustration of a medical procedure that utilizes elongate members such as angioplasty. Entry via femoral artery is most common, however, the site of entry may be at the brachial artery, the subclavian artery, or any other suitable anatomical point.

FIG. 2 shows an embodiment of a computer-assisted system for manipulating elongate members in a medical procedure.

FIG. 3 illustrates a block diagram of a computer system that can be utilized in association with embodiments of the invention.

FIGS. 4A and 4B show an embodiment of a module base that can be utilized to advance, retract, rotate, and retain elongate members.

FIG. 5 shows wheels that can be driven to advance or retract elongate members.

FIG. 6 shows a flow chart of a process of the initial placement of the guide catheter.

FIG. 7 shows a flow chart of a process of placing the guide wire at the desired destination.

FIG. 8 shows a flow chart of a process of utilizing interventional catheters.

FIG. 9 shows a drum that can be utilized to retract one elongate member while maintaining the position of one or more other elongate members.

FIG. 10 shows a flow chart of a process of retracting an interventional catheter.

FIGS. 11A and 11B show another embodiment of a module base that can be utilized to advance, retract, rotate, and retain elongate members.

FIG. 12 shows another embodiment of a computer-assisted system for manipulating elongate members in a medical procedure where the module base moves on rails in a helical arrangement.

FIG. 13 shows a screen image of a menu where a user can select the mode for manipulating the elongate members.

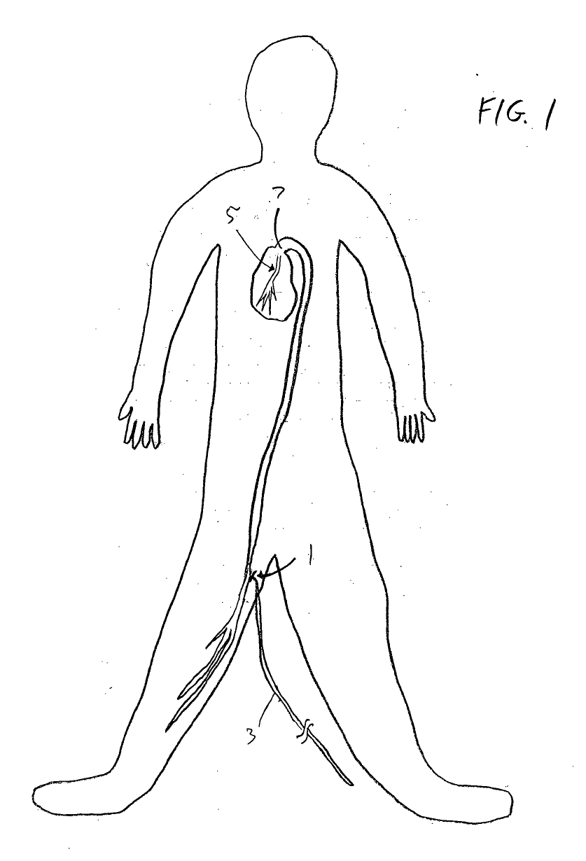

### DETAILED DESCRIPTION OF PREFERRED EMBODIMENTS

In the description that follows, the present invention will be described in reference to embodiments that utilize computers and electromechanical devices to manipulate elongate members such as catheters and guide wires. More specifically, the embodiments will be described in reference to preferred embodiments. However, embodiments of the invention are limited to any particular configuration, architecture, or specific implementation. Therefore, the description of the embodiments that follows is for purposes of illustration and not limitation.

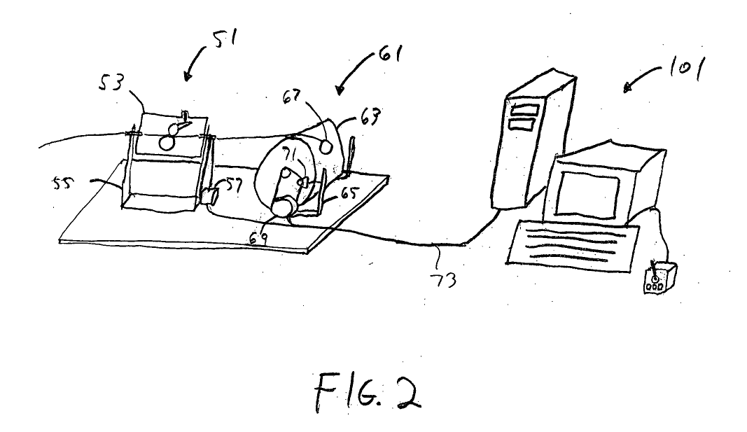

FIG. 2 shows a system of one embodiment of the invention that provides computer-assisted manipulation of elongate members in medical procedures. A module base assembly 51 is proximal to the patient and allows for advancement, retraction, rotation, and retention of elongate members. Module base assembly 51 includes a module base 53 that is supported by a stand 55.

Module base 53 may be rotated along the axis of the elongate members and can be driven by a stepper motor 57. Motor 57 can be coupled to module base 53 for driving rotation utilizing a belt, chain, worm gear drive or similar components. A gearbox may be interposed between the stepper motor and the gear that rotates the module base.

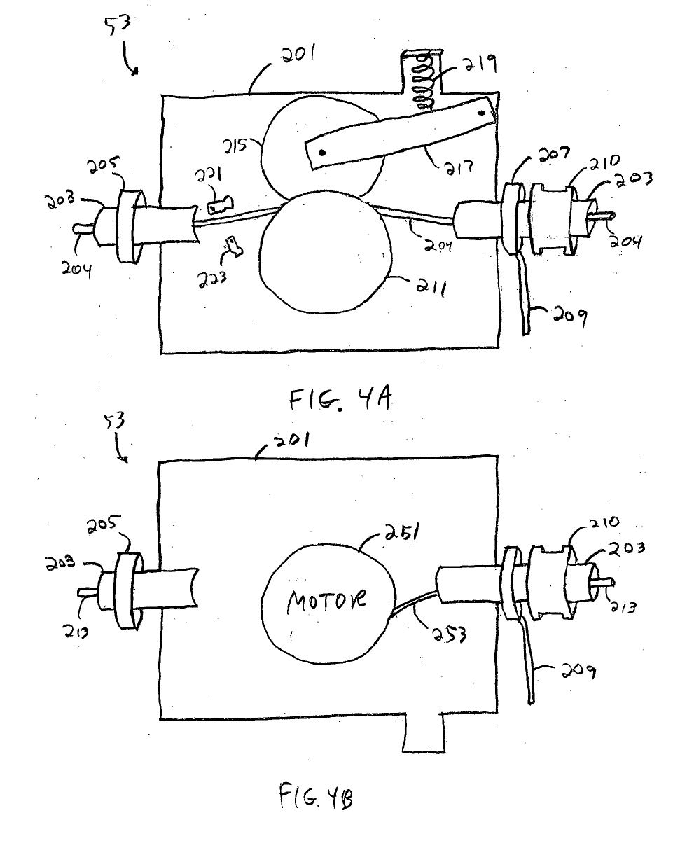

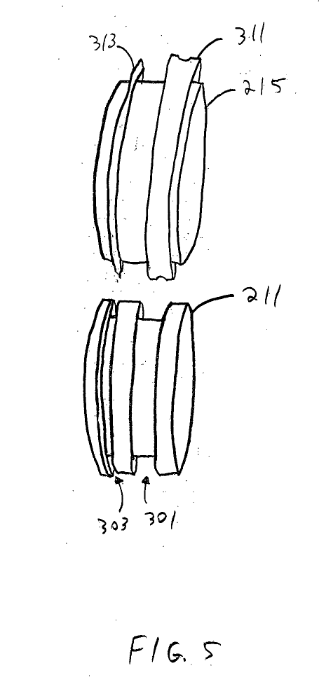

Module base 53 includes a motor and at least one wheel for advancing or retracting the elongate members. A specific embodiment of module base 53 will be described in more detail in reference FIGS. 4A and 4B.

A drum assembly 61 can be utilized to advance or retract a flexible elongate member while maintaining pressure on one or more other elongate members so that they stay in their current and desired location. Drum assembly 61 includes a drum 63 that is supported by a stand 65. A wheel (or spool) 67 is located on drum 63 and can advance or retract elongate members. Wheel 67 can be driven by a motor that is positioned inside drum 63 (not shown).

Drum 63 rotates along an axis that is perpendicular to the elongate members. Accordingly, when drum 63 rotates, elongate members that are attached to the drum can be advanced or retracted. Rotation of the drum can be driven by a motor 69 utilizing a belt as shown. Additionally, a biasing mechanism 71 can be attached to stand 65 in order maintain the proper tension of the belt. Alternatively, motor 69 can drive the drum via chain, worm drive, or other standard drive components, with or without an interposed gearbox.

Although drum assembly 61 can be directed by the computer system to operate in many different ways, drum assembly 61 can be utilized to retract an elongate member while maintaining the other elongate members at their current locations. For example, the elongate member to be retracted can be secured to drum 63, which is then driven to retract the elongate member. At the same time, wheel 67 can be driven advance one or more other elongate members, for example a guide wire, so that they stay at their current desired location. More details on a specific embodiment of drum 63 will be described in more reference to FIG. 9.

Wires 73 connect the various electromechanical devices to a computer system 101. Computer system 101 is directed by a user and then utilizes electrical signals in order to control the electromechanical devices. The user can direct computer system 101 through common types of input such as a keyboard and a pointing device (e.g., joystick as shown, mouse, trackball, and the like). In addition to continuous movement, a mode of operation can be included to individually step or “bump” the controlled device or devices in movements of very fine resolution or granularity. This may be done in a rapid “hammering” fashion, or slowly and gently, in accordance with the needs for member advancement or retraction. Thus, the operator can achieve greater control than he or she would have while using traditional manipulation techniques.

FIG. 2 shows module base assembly 51 drum assembly 61 and computer system 101 that are capable of providing computer-assisted manipulation of elongate members during medical procedures. However, systems of the invention are not limited by the number of components. For example, a system can include fewer components, like module assembly 51 and computer system 101 or drum assembly 61 and computer system 101. Additionally, other embodiments can utilize additional components. For example, a system can include two (or more) module base assemblies similar to the one show in FIG. 2. In this manner, a first module base assembly can manipulate (e.g., advance, retract, rotate, or retain) one elongate member while a second modular base assembly manipulates a second elongate member. Thus, with two module base assemblies, the guide catheter and guide wire could both be manipulated simultaneously.

In an alternative embodiment, a stepper motor-driven rotation system located on the module base can drive the rotation of an individual device member. In yet another embodiment, linear actuators upon which gripper mechanisms are attached servers to grasp each wire or catheter, move it to the end of that linear actuator's travel, release their grip on the wire or catheter, return to their opposite extreme position, re-grip the wire or catheter, and begin another step of moving the wire or catheter to its intended location. The physical shape and layout of the member-driving apparatus may also be cantilevered; securely attached to the side of the operating table or other platform, and extending over the top of the patient's body. Such a configuration may be desirable in order to facilitate the introduction of catheters and wires at selected entry points to the body.

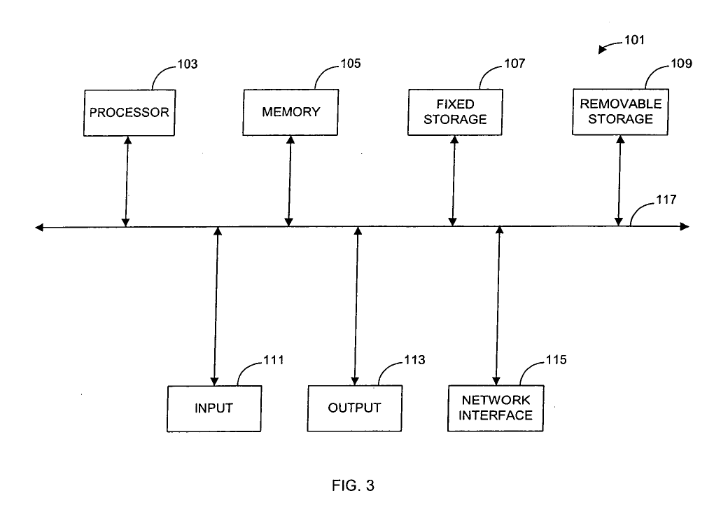

FIG. 3 shows a block diagram of components that can be present in computer systems that implement embodiments of the invention. A computer system 101 includes a processor 103 that executes instructions from computer programs (including operating systems). Although processors typically have memory caches also, processor 103 utilizes memory 105, which can store instructions (or computer code) and data.

A fixed storage 107 can store computer programs and data such that it is typically persistent and provides more storage when compared to memory 105. A removable storage 109 provides mobility to computer programs and/or data that are stored thereon. Examples of removable storage are floppy disks, tape, CD/ROM, flash memory devices, and the like.

Memory 103, fixed storage 107 and removable storage 109 provide examples of computer readable storage media that can be utilized to store and retrieve computer programs incorporating computer codes that implement the invention, data for use with the invention, and the like. Additionally, a data signal embodied in a carrier wave (e.g., in a network including the Internet) can be the computer readable storage medium. An input 111 allows a user to interface with the system. Input can be done through the use of a joystick, keyboard, a mouse, buttons, dials, or any other input mechanism. An output 113 allows the system to provide output to the user. Output can be provided through a monitor, display screen, LEDs, printer or any other output mechanism.

A network interface 115 allows the system to interface with a network to which it is connected. The system bus architecture of computer system 101 is represented by arrows 117. The components shown in FIG. 3 can be found in many computer systems. However, components can be added, deleted and combined. For example, fixed storage 107 could be a file server that is accessed through a network connection. Thus, FIG. 3 is for illustration purposes and not limitation.

FIGS. 4A and 4B show an embodiment of the module base that can manipulate one or more elongate members. A plate 201 serves as the foundation for other components. Two pipes 203 are attached on opposing ends of plate 201. One or more elongate members 204 pass through the lumen of pipes 203.

Plate 201 rotates about an axis provided by pipes 203. A rotating member 205 is coupled to one of pipes 203. A slip ring connector 207 is coupled to one of pipes 203, allowing electrical signals to be sent to electromechanical devices on plate 201 over one ore more wires 209, such that as plate 201 rotates, the one or more wires 209 do not coil up on pipe 203.

In an alternative embodiment, pipe 303 may be present only on the right (proximal) side of plate 201, thus allowing plate 201 to be shorter, and for wheel 211 to be closer to the site of elongate member entry into the body, thus requiring less elongate member length.

Pulley 210 is coupled to pipe 203 as shown. Referring back to FIG. 2, motor 57 drives a belt that is in contact with pulley 210 in order to rotate plate 201.

A wheel 211 is rotateably coupled to plate 201. Wheel 211 has one or more grooves for accepting elongate members 204 so that they can be advanced or retracted depending on the rotation of wheel 211. Wheel 211 is driven by a motor that is positioned on the other side of plate 201 and will be described in more detail in reference to FIG. 4B. Additionally, more details on wheel 211 will be described in reference to FIG. 5.

A wheel 215 is utilized to maintain friction between wheel 211 and elongate member 204. As shown, wheel 215 rotates on a fulcrum 217 and is biased against wheel 211 by a spring 219. Although this biasing mechanism is shown, other biasing mechanisms may be utilized. More details on wheel 215 will also be described in relation to FIG. 5.

Clips 221 and 223 allow for an elongate member to be retained on plate 201. In other words, the clip can be utilized to hold the elongate member so that it will not be advanced or retracted. Clips 221 and 223 can be of different sizes in order to retain different types of elongate members.

These clips, and other forms of releasable fixation devices, may be operated manually or may be operated via motorized remote control. For example, the catheter or wire may be releasably fixed at a certain point by the activation of a solenoid or electromechanical gripper, as is known in the art. The activation of this solenoid or gripper may be mediated by the software and activated by direct designation on the interface, the joystick, associated buttons, or automatically as part of entering one of several movement modes provided by the software, or automatically upon the completion of specific movement sequences. Alternatively, the closing and opening of a solenoid-based clip may be accomplished by the direct, manual action upon an electrical switch.

FIG. 4B shows the opposite side of plate 201 from FIG. 4A. As shown, a motor 251 is coupled to plate 201 opposite wheel 211. Electrical signals passed to motor 211 through one or more wires 253.

As shown, module base 53 in FIGS. 4A and 4B allow for elongate members to be advanced, retracted, rotated, and retained. The elongate members can vary in size so it may be beneficial to describe embodiments of wheel 211 and bias wheel 215 that accommodate elongate members with varying diameters. FIG. 5 shows a side view of wheel 211 and bias wheel 215 shown in FIG. 4A. Some elongate members like the guide or interventional catheters have a greater diameter than the guide wire. Elongate members with larger diameters can be threaded through a larger groove 301 on wheel 211. Bias wheel 215 has a corresponding structure 311 that includes a groove such that when bias wheel 215 is pressed against wheel 211, friction is maintained between the elongate member and wheel 211.

An elongate member with a smaller diameter can be placed in a smaller groove 303. A corresponding structure 313 on bias wheel 215 helps maintain friction between the elongate member and wheel 211 when bias wheel 215 is biased against 211. The specific grooves and other mechanisms for maintaining friction on the elongate members can be varied in other embodiments. The friction of wheel 211 against the elongate member causes the elongate member to be moved in accordance with the movement of wheel 211. This also applies when wheel 211 is rotated perpendicular to its normal axis of spin. Friction may be increased by increasing spring tension, by using multiple wheels 211 in series, multiple bias wheels 215 in series, and by using caterpillar treads between wheel 211 and another active or passive wheel that moves in tandem with wheel 211.

The elongate members can be flexible or rigid. Many examples of flexible members have been described. Additionally, the invention can be advantageously applied to manipulate rigid members, such as biopsy needles. Therefore, the invention is not limited by the specific embodiments shown.

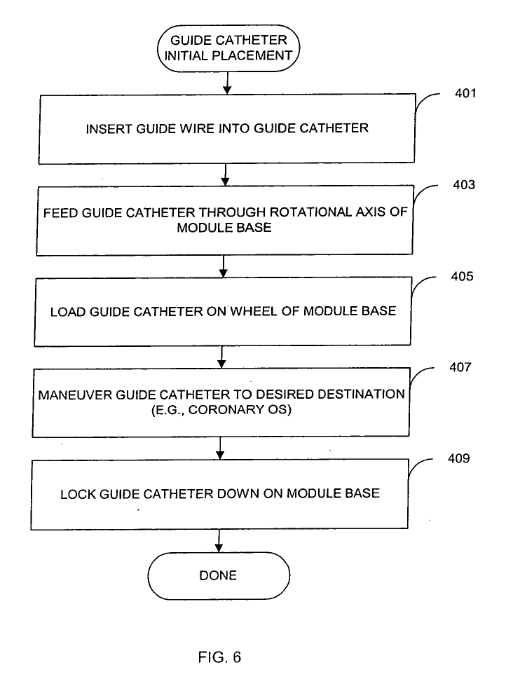

Now that embodiments of an overall system have been described, it may be beneficial to describe procedures utilizing the system. FIG. 6 shows a flow chart of a process of the initial placement of a guide catheter. As with all flow charts shown herein, steps may be added, deleted, combined, or reordered without departing from the spirit and scope of the invention. At a step 401, the guide wire is inserted into the guide catheter. Typically, the guide catheter has a slightly bend end that allows it to be maneuvered into the desired destination. For example, as described above, the guide catheter can be maneuvered to the coronary os.

The guide catheter is fed through the rotational axis of the module base at a step 403. Referring back to FIG. 4A, the guide catheter can be inserted into pipes 203.

At a step 405, the guide catheter is loaded on the wheel of the module base. Once again, referring back to FIG. 4A, wheel 215 can be retracted so that the guide catheter can be placed in groove 301 on wheel 211. At this point, the guide catheter can be maneuvered utilizing computer-assisted control to the desired destination within the patient.

The guide catheter is maneuvered to the desired destination at a step 407. A pointing device can be utilized to advance, retract, and rotate the guide catheter as it is maneuvered to the desired destination, such as the coronary os (or opening). For example, on a joystick, “forward” can advance, “backward” can retract and “left/right” can rotate in the respective direction. As is common in the art, imaging modalities such as fluoroscopy, ultrasound, real-time computerized tomography, or magnetic resonance imaging can be utilized to assist the user in maneuvering the guide catheter.

Once the guide catheter has reached the desired destination, the guide catheter is locked down on the module base at a step 409. For example, clip 221 shown in FIG. 4A can be utilized to retain the guide catheter on plate 201. Clip 221 should be placed near the end of the guide catheter so that module base 53 can be utilized to maneuver the guide wire or interventional catheter. As mentioned before, a second (or multiple) module base assembly can be utilized to independently maneuver one or more elongate members.

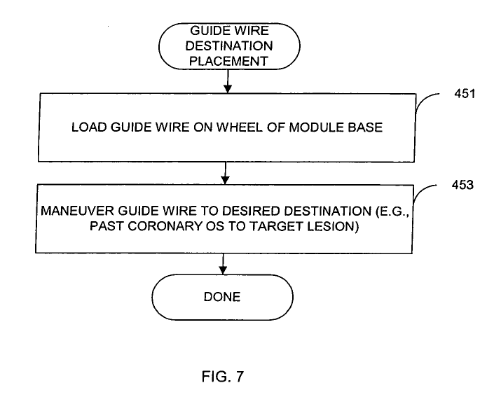

FIG. 7 shows a flow chart of a process of placing the guide wire at the desired destination. At a step 451, the guide wire is loaded on the wheel of the module base. Referring to FIG. 4A, wheel 215 may be retracted to allow the guide wire to be loaded in groove 303 of wheel 211.

The guide wire is maneuvered to the desired destination at a step 453. For example, module base 53 can be directed to maneuver the guide wire from the coronary os to the target lesion. Now that the guide wire is at the desired destination, and the interventional catheter can be utilized to complete the medical procedure.

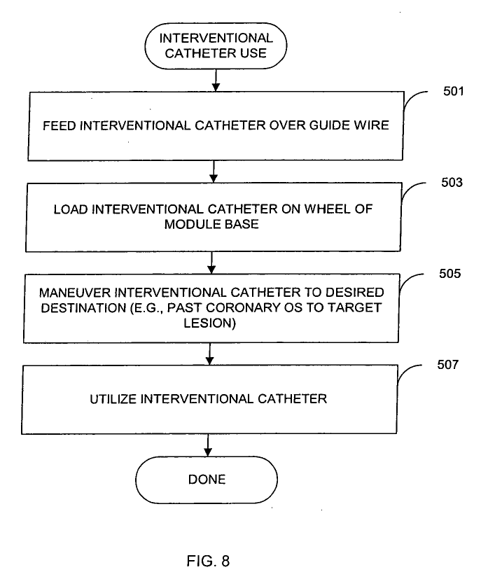

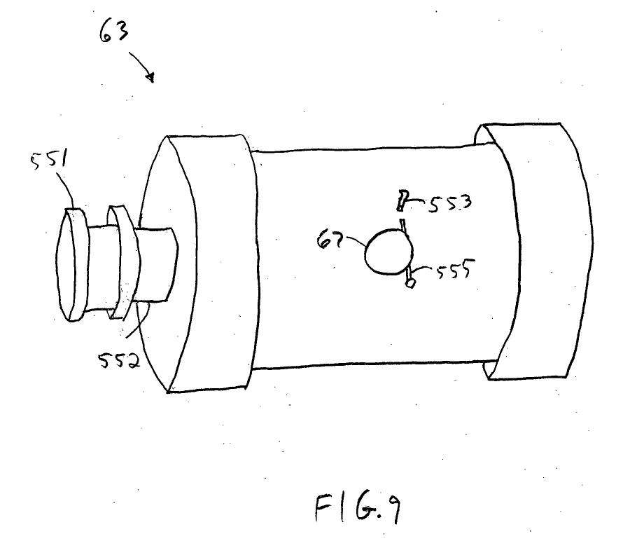

FIG. 8 shows a flow chart of a process of utilizing an interventional catheter. The interventional catheter basically is fed over the guide wire and within the guide catheter. The interventional catheter is loaded on the wheel of the module base at a step 503. The module base will be utilized to maneuver the interventional catheter to the desired destination.

At a step 505, the interventional catheter is maneuvered to the desired destination. For example, typically the interventional catheter is maneuvered through the guide catheter to the coronary os and then follows the path set out by the guide wire to the target lesion.

The interventional catheter is utilized at a step 507. As described above, the interventional catheter can be an angioplasty catheter that inflates a balloon or a stent catheter that inserts a stent. Other types of interventional catheters can also be advantageously utilized with embodiments of the present invention.

In many medical procedures of this type, it may be beneficial to swap out the interventional catheter being utilized. One of the disadvantages of prior techniques is when an interventional catheter is retracted, the friction on the other elongate members can result in the other elongate members (e.g., guide catheter) being retracted away from their desired destination. Drum assembly 61 can be utilized to reduce or eliminate the possibility of this occurrence.

FIG. 9 shows the drum of the drum assembly. The drum can be rotated about its axis by a motor that drives a belt that is coupled to pulley 552. Alternatively, the apparatus may be driven by a chain or worm gear, with or without an interposed gearbox. A slip ring electrical connector (with no bore hole) 552 allows electrical connections to pass to and from rotating drum 63.

Drum 63 includes a clip 553 for retaining elongate members, such as an interventional catheter. As with the clips described previously, these releasable fixation devices maybe electronically activated. As described above, wheel 67 is driven by a motor that is located within the drum (not shown). Wheel 67 can operate as a spool that stores an elongate member such as the guide wire, or may simply pull wire behind it, in the manner of wheel 211 Regardless, wheel 67 can act to advance or retract an elongate member and a biasing member 555 can be utilized to maintain friction wheel 67 and the elongate member. As shown, biasing mechanism 55 is a strip of spring steel metal that is biased against wheel 67. Optionally, a bias wheel like bias wheel 211 may be used as the method for keeping the elongate member in frictional contact with the drive mechanism. In an alternative embodiment, a small hole may be provided through the surface of drum 63 so that the tail end of wire retracted by wheel 67 can coil itself in the hollow interior drum 63. Other containers affixed to the drum may also be used to hold the tail end of the guide wire.

As will be described below, drum 63 can be rotated to retract a first elongate member while wheel 67 is directed advance a second elongate member. This allows the first elongate member to be retracted without moving the second elongate member from its desired location.

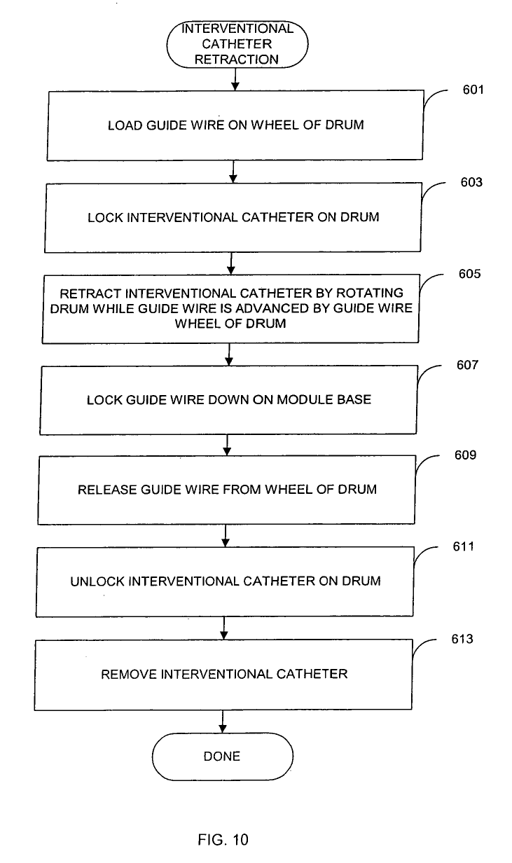

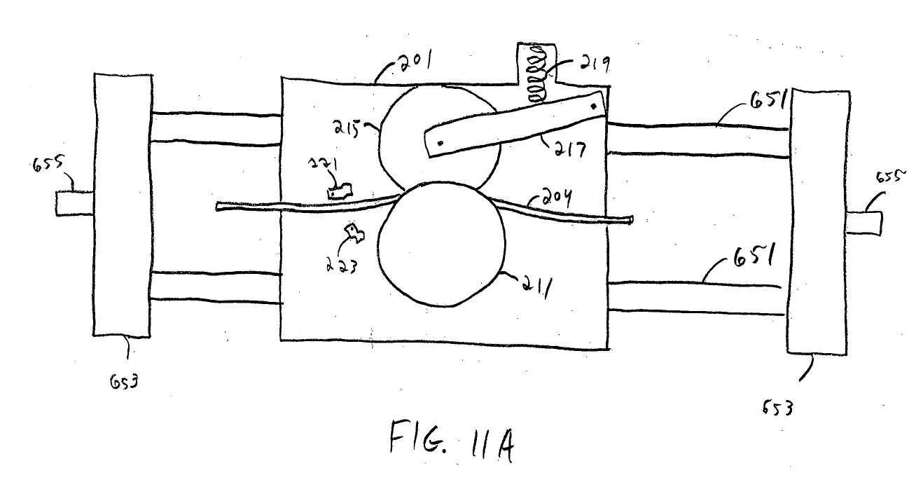

Now that drum 63 has been described in more detail, it may be beneficial to describe how the drum assembly can be utilized to retract an interventional catheter. FIG. 10 shows a flow chart of a process of retracting an interventional catheter

At a step 601, the guide wire is loaded on the wheel of the drum. Referring back to FIG. 9, the guide wire is loaded on wheel 67 of drum 63. When wheel 67 is rotated backwards, the guide wire will spool up on wheel 67, or be driven into the interior of the drum, or drum-associated container, so that when drum 63 rotates, the distal portion of the guide wire will not be in the way. If wheel 67 is not engaged, or if the drum does not need to be rotated, the guide wire may be left to freely trail over and behind the drum.

The interventional catheter is then locked on the drum at step 603. For example, the interventional catheter can be retained on the drum utilizing clip 553.

At step 605, the interventional catheter is retracted by rotating the drum while the guide wire advanced by the wheel of the drum. When the user instructs the computer system to retract the interventional catheter, motor 69 directs rotation of drum 63 to retract the interventional catheter while at the same time the motor coupled to wheel 67 is directed to advance the guide wire. As wheel 67 is coupled to drum 63, the rotation of drum 63 acts to retract the guide wire. However, the rotation of wheel 67 advances the guide wire and is preferably controlled so that the advancement counters the retraction of the guide wire so that the guide wire stays at the desired location.

The guild wire on wheel 67 may be advanced through the guide catheter or interventional catheter without buckling, provided that the opening to the guide catheter or interventional catheter is held firmly in place by clip 553 and in close proximity to the exit of the path from wheel 67 and clip 553. This ability to maintain the guide wire in place, despite the retraction of adjacent or surrounding members, may be very advantageous since threading it through a region of blockage can be a delicate procedure and one that one would preferably not want to repeat.

Once the interventional catheter is retracted past or to module base 53, the guide wire is locked down on the module base at a step 607. For example, clip 223 on plate 201 can be utilized to retain the guide wire.

At a step 609, the guide wire can be released from the wheel of the drum. For example, wheel 67 can be rotated to unspool the guide wire from wheel 67, or to drive it forward from its extended or coiled resting point behind wheel 67.

The interventional catheter can be unlocked on the drum at a step 611. For example, the end of interventional catheter can be released from clip 553 on drum 63. Now, the interventional catheter can be removed at a step 613 by pulling it off the guide wire.

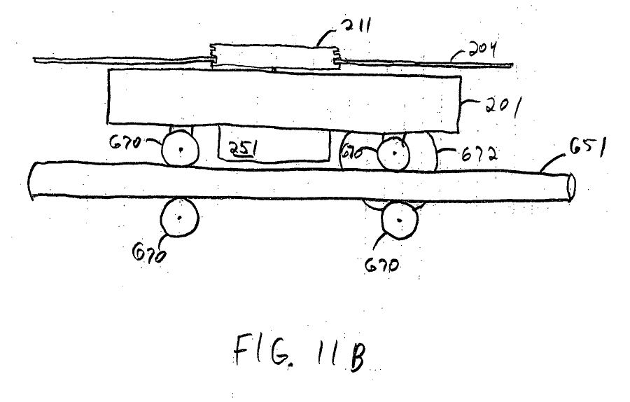

FIG. 11A shows another embodiment of a module base. The module base is similar to what is shown and described in reference to FIGS. 4A and 4B. However, in this embodiment, module base 201 is movably mounted to two rails 651. The rails are held stable by two end plates 653. The assembly shown in FIG. 11B can be rotated by a motor and pivot about end points 655.

The module base moves along rails 651 as shown in FIG. 11B. Wheels 670 ride on rails 651 and are biased against the rails in pairs as shown. In order to drive module base to advance or retract, a motor 672 drives one of the wheels.

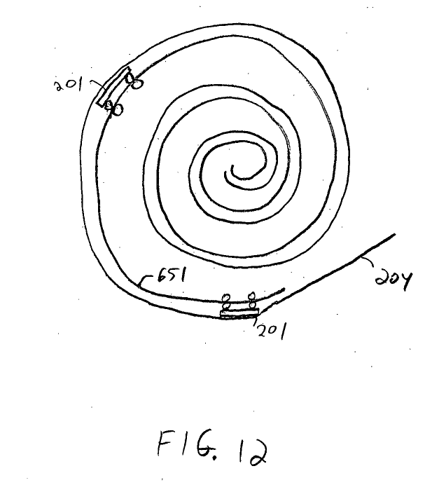

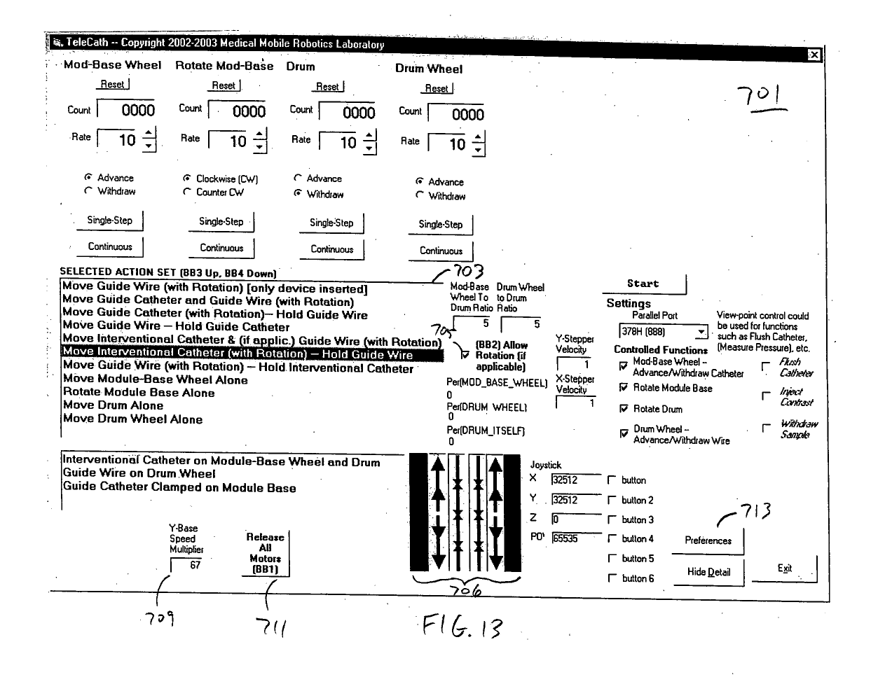

A system that utilizes the module base assembly shown in FIGS. 11A and 11B can be utilized in a manner similar to what has been described above. If it is desirable to reduce the size of the system (e.g., the rails may extend several feet or more), an embodiment can be utilized as shown in FIG. 12.

FIG. 12 shows another embodiment of a computer-assisted system for manipulating elongate members in a medical procedure where the module base moves on rails in a helical arrangement. Rail 651 are coiled in a helical (or nautilus) arrangement. One or more module bases 201 can be directed by a computer system to move along the rails, advance or retract elongate member 204, and the like. In order to rotate elongate member 204, the entire helical structure can be rotated by a motor. Alternatively, the drive wheel on each module base may be independently rotated, for example, by additional, module-base-specific step motors.

As described above, a user can utilize a pointing device to direct manipulation of the elongate members. A graphical user interface can also be utilized to direct the operation of the system.

FIG. 13 shows an example of a graphical user interface menu that directs the operation of the system. A window 701 includes a menu 703. Menu 703 allows the user to select the operational mode of the system, including which the axes of movement are to be active and under control of the joystick at any given time. A mode dictates which motors on the apparatus will be active concurrently, in which direction they will move, and at what relative speeds. For example, the selection that has been made directs the system to move the guide catheter and guide wire with rotation.

In one embodiment, the mode can be selected by pressing buttons on a joystick base. Another method can be to select the desired mode on the screen by positioning a cursor with a pointing device such as a mouse, track pad, track ball, or the like, and selecting with a button click.

The operator can be informed as to the type or types of motion anticipated in the various modes by a graphic area 706 displayed on a portion of the screen, with the graphics changing depending on which mode is selected. For example, such a graphic may include a longitudinal cross-sectional diagram of a guide catheter with an interventional catheter and a guide wire within. Figures such as arrows (for motion) and X's (for stasis) may be graphically superimposed upon the appropriate parts of the cross-sectional diagram, thus showing what elements will be moved and what elements will remain still at each selected motor combination mode.

A check box 707 allows a user to specify whether to allow rotation in a selected mode if it is applicable. A text window 707 shows the current status of the system. A user is able to enter a speed multiplier in text box 709. This speed multiplier, in conjunction with direction and speed input information output by switches and the angular displacement of the joystick, will affect the speed at which the elongate members are manipulated, such as advanced and retracted.

In one embodiment, the speed of stepper movement is proportional to the deviation (or movement) from neutral of the joystick handle (or other pointing device). Small deviations from neutral may be ignored in order to promote stability, ease of use, and to prevent accidental movement triggering. Setting the differential speed between the various motorized components is a function that is governed by the controlling software and is advantageous for providing coordinated actions.

A button 711 allows all the motors to be released. By releasing the motors, the elongate members in contact with the wheel, which are coupled to the motors, will provide minimal resistance to the elongate members. This may be accomplished in order to facilitate changing catheters and wires in and out of the various drive mechanisms. A button 713 allows a user to specify preferences for the system. Alternatively, or in addition to, physical buttons on the joystick may be used to set preferences for the system, including stepper combination mode, speed, stepper release, etc.

While the above is a complete description of preferred embodiments of the invention, various alternatives, modifications, and equivalents can be used. It should be evident that the invention is equally applicable by making appropriate modifications to the embodiments described. Therefore, the above description should be taken as limiting the scope of the invention that is defined by the metes and bounds of the appended claims along with their full scope of equivalents.

1. A method of manipulating an elongate member during a medical procedure, comprising:
receiving input from a user to manipulate the elongate member;
sending signals to advance the elongate member if the input directs advancement of the elongate member;
sending signals to retract the elongate member if the input directs retraction of the elongate member; and
sending signals to rotate the elongate member if the input directs rotation of the elongate member.

2. The method of claim 1, wherein the elongate member is flexible or rigid.

3. The method of claim 1, wherein the signals specify a speed that is proportional to movement of a pointing device.

4. The method of claim 1, wherein the input is received from a pointing device coupled to a computer system.

5. The method of claim 1, wherein the signals to advance the elongate member direct a motor to rotate a wheel in contact with the elongate member.

6. The method of claim 1, wherein the signals to retract the elongate member direct a motor to rotate a wheel in contact with the elongate member.

7. The method of claim 1, wherein the signals to rotate the elongate member direct a motor to rotate a base that is coupled to the elongate member.

8. An apparatus for manipulating elongate members during medical procedures, comprising:
a base coupled to an elongate member, the base being capable of rotation along an axis parallel to the elongate member;
a first motor coupled to the base that advances or retracts the elongate member along the axis; and
a second motor coupled to the base that rotates the base, whereby the elongate member is rotated around the axis.

9. The apparatus of claim 8, wherein the relative speed of the first and second motors provides coordinated motion.

10. The apparatus of claim 8, wherein the first motor advances or retracts the elongate member by rotating a wheel in contact with the elongate member.

11. The apparatus of claim 10, further comprising a biasing mechanism to bias the elongate member against the wheel.

12. The apparatus of claim 8, further comprising a clip to retain the elongate member.

13. The apparatus of claim 8, further comprising a computer system that receives user input to direct the first and second motors.

14. A method of manipulating an elongate member during a medical procedure, comprising:
retracting two elongate members where a first elongate member is within the lumen of a second elongate member; and
advancing the first elongate member relative to the second elongate member.

15. The method of claim 14, wherein the first elongate member is advanced to substantially counter retraction caused by the retraction.

16. An apparatus for manipulating elongate members during medical procedures, comprising:
a drum coupled to two elongate members where a first elongate member is within the lumen of a second elongate member, the drum being rotatable along an axis perpendicular to the two elongate members and comprising a clip to retain the second elongate member such that when the drum rotates, the second elongate member is retracted along a first direction; and
a wheel coupled to the drum such that the rotation of the drum also rotates the wheel and the first elongate member is retracted along the first direction, wherein the wheel rotates to advance the first elongate member along a second direction opposite the first direction.

17. The apparatus of claim 16, wherein the elongate member is advanced to substantially counter retraction caused by rotation of the drum.

18. The apparatus of claim 16, further comprising a motor coupled to the drum that rotates the drum.

19. The apparatus of claim 16, further comprising a motor coupled to the wheel that rotates the drum.

20. The apparatus of claim 16, further comprising a computer system that receives user input to direct rotation of the drum, wheel or both.
